(19) United States
(12) Patent Application Publication
(10) Pub. No.: US 2007/0207499 A1
(43) Pub. Date: Sep. 6, 2007

(54) Method for measuring a protein

(75) Inventors: Katsuyoshi Takahashi, Tokyo (JP); Kiyohito Shimura, Sagamihara-shi (JP); Haruo Matsuda, Hiroshima (JP); Masayoshi Aosasa, Hiroshima (JP)

Correspondence Address: NIXON & VANDERHYE, PC, 901 NORTH GLEBE ROAD, 11TH FLOOR, ARLINGTON, VA, 22203, US

(73) Assignees: Sharp Kabushiki Kaisha, Osaka (JP); TEIKYO UNIVERSITY, Tokyo (JP)

(21) Appl. No.: 11/516,057

(22) Filed: Sep. 6, 2006

(30) Foreign Application Priority Data
Sep. 6, 2005 (JP) 2005-257938

(51) Int. Cl. G01N 33/53; C12Q 1/37; C12M 3/00

(52) U.S. Cl. 435/7.1; 435/23; 435/287.2

(57) ABSTRACT

The present invention provides a method for measuring a particular protein in a sample containing at least one protein, wherein the sample is reacted with a reagent cleaving a peptide bond of the particular protein to generate a soluble peptide fragment which is determined by a certain primary structure; and contacted with a reagent reacting specifically with the particular soluble peptide fragment, thereby detecting the presence of the particular soluble peptide fragment.

### CROSS-REFERENCE TO RELATED APPLICATION

This application is related to Japanese Patent Application No. 2005-257938 filed on Sep. 6, 2005, whose priory is claimed under 35 USC § 119, the disclosure of which is incorporated herein in its entirety by reference for any and all purposes.

### BACKGROUND OF THE INVENTION

1. Field of the Invention

The present invention relates to a method for measuring a protein after degrading the same into peptide fragments.

2. Description of the Related Art

Recently, much attention has been focused on such a technology that measures a certain particular protein from among a population of proteins, which are biological substances and of various kinds.

The measurement of a particular protein has significance in utilizing the amount of a particular protein related to a disease for diagnosis or prevention of the disease, the amount of a particular harmful protein in an environment or food for the assessment of the environment or food, or the like. Also, the measurement of a particular protein makes it possible to know the effects of administrating an agent on a protein.

(Conventional Method for Measuring a Protein)

Methods for measuring a particular protein include, for example, immunoassays such as immunoassay coupled with fluid control including the ELISA method described in Japanese Laid-Open Patent Publication No. 2002-207043, the affinity electrophoresis described in International Publication No. WO 94/17409, the Western blot method described in European Patent Publication No. 0 397 129 A2, and the like. In immunoassays, a particular protein (such as antigen) of interest is measured using a protein (such as antibody) that binds specifically with the particular protein. Here, the antibody is required to be capable of forming a stable immunological complex specifically with the particular protein. An affinity substance which binds specifically with a particular protein (such as antigen) is not limited to a protein (such as antibody). It may be a peptide, a nucleic acid, synthetic chemicals, or the like. In any immunoassays, it is necessary to maintain the binding activity that is involved in the formation of a complex between an antigen and an affinity substance therefor such as an antibody during the measurement.

In ELISA (Enzyme Linked Immunosorbent Assay) methods, a primary antibody protein having the capability of specifically binding with an antigen protein of interest is immobilized to a solid support, the support is blocked to prevent any nonspecific adsorption of the antigen, and then a sample containing the antigen protein of interest is added. After the binding reaction occurs between the antigen protein and the primary antibody protein, proteins not reacted with the primary antibody protein are removed by washing. Then, a labeled secondary antibody, which binds specifically to a site in the antigen protein different from the site that is bound by the primary antibody protein, is added and permitted to bind. It is general to use an enzyme, a fluorescent dye, a chemical chromophore, or the like as a label conjugated to a secondary antibody. After the unreacted, labeled secondary antibody is removed by washing, the amount of the antigen protein in the sample is determined based on a signal from the enzymatic reaction by the addition of a substrate for the enzyme, a signal from the fluorescent dye, or a signal from the chemical chromophore. In ELISA methods, an antigen, a primary antibody, and a secondary antibody used are all required to be soluble, and it is necessary to maintain the binding activity of the antigen and the antibody during the reaction process.

For affinity electrophoresis, it is possible to use, as a mode of separation, a zone electrophoresis which separates proteins based predominantly on their electric charge, an isoelectric focusing electrophoresis which separates proteins based on the difference in the isoelectric point therebetween, and a molecular sieve gel electrophoresis which separates proteins based on the difference between their molecular weights. In any of the separation modes, the presence/absence and the amount of the antigen are determined based on the difference in the electrophoretic separation pattern between unbound labeled antibody and the complex of the antigen with the labeled antibody. Generally, a fluorescent dye is used as a label. The antigen and the antibody to be used in electrophoresis are required to be soluble, and it is desirable that the antigen-labeled antibody complex and the unbound labeled antibody are each detected as a single peak.

In Western blot method, first, a sample containing antigen proteins is separated by gel electrophoresis based on the molecular weight of the proteins. In gel electrophoresis, SDS-PAGE is commonly used. In SDS-PAGE, the proteins in a sample are treated with SDS (Sodium Dodecyl Sulfate), an anionic surfactant, and mercaptoethanol, a reducing agent, so that the higher-order structure of each protein is destroyed and all the proteins are negatively charged, and the proteins are separated by the sieve effect of polyacrylamide gel based on the difference in the molecular weight between the proteins. The separated proteins are transferred from the gel into a membrane such as PVDF by applying an electric current. After transferring, the surfactant and the reducing agent are removed so that the membrane is in conditions that allow the reaction of antigens and antibodies to occur, and then the membrane is blocked. After blocking, a solution of a labeled antibody specifically binding with the antigen of interest is added and the binding reaction is permitted. It is common to use an enzyme, a fluorescent dye, a chemical chromophore, or the like as a label conjugated to an antibody in this technique. After the unreacted, labeled antibody is removed by washing, the amount of the antigen protein in the sample is determined based on a signal from the enzymatic reaction by the addition of a substrate for the enzyme, a signal from the fluorescent dye or a signal from the chemical chromophore.

In Western blot method, it is necessary to solubilize a particular protein of interest into a solution containing a disulfide bond reducing agent such as SDS and mercaptoethanol before acrylamide gel electrophoresis is conducted.

In some assays, for example, a peptide, a nucleic acid, or a synthetic chemical can be used as an affinity substance, instead of an antibody protein described above.

(Causes of the Insolubility of Insoluble Proteins)

First of all, the existence of an amino acid residue having a hydrophobic side group, including alanine, valine, leucine, isoleucine, proline, methionine, phenylalanine, and tryptophan, is mentioned as one of the causes of the insolubility of insoluble proteins. In a usual soluble protein, hydrophobic amino acid residues are folded inside in an aqueous solution so that they make little contact with the aqueous solution, and in the interface with the aqueous solution, a lot of hydrophilic amino acid residues are arrayed, and consequently the soluble protein as a whole is solubilized in the aqueous solution. A protein in which many hydrophobic amino acid residues cannot be folded inside is insoluble. In an insoluble protein such as a membrane protein, its hydrophobic parts are bound with the lipid parts of the phospholipid of the associated membrane, and as a result, such a protein is present stably in such a manner that its some (hydrophobic) parts are buried into the membrane. When the phospholipid is removed, the hydrophobic parts are exposed to the interface with the ambient aqueous environment, and therefore the hydrophobic parts bind with each other and the proteins aggregate and become insoluble.

Also mentioned are aggregation of proteins via disulfide bond between cysteine residues of any two of the proteins, or via noncovalent bond such as hydrogen bond, electrostatic interaction, hydrophobic interaction, and van der Waals force, and binding of a protein with an insoluble substance such as a lipid.

A covalent bond between amino acid residues of proteins may also make the proteins insoluble. This can be seen in, for example, crosslinked collagen or elastin (which is crosslinked by dehydrolysinonorleucine, desmosine, isodesmosine, histidinohydroxymerodesmosine, or the like), polymerized fibrin (which is crosslinked through isopeptide bond).

(Conventional Methods for Solubilizing an Insoluble Protein)

Conventional methods for solubilizing an insoluble protein not dissolving in a physiological salt solution are as follows.

Methods for solubilizing a protein having hydrophobic amino acid residues on the surface include, for example, a method of solubilizing such a protein by adding a surfactant such as SDS, Triton X, or the like. In the case where the insolubility is due to the disulfide bond between cysteine residues, the insoluble protein can be solubilized by adding a reducing agent, such as, for example, mercaptoethanol and dithiothreitol, to cleave the disulfide bond. In the case where a protein aggregates through noncovalent bond such as hydrogen bond, electrostatic interaction, hydrophobic interaction, and van der Waals force, and thus is insoluble, such a protein can be solubilized by adding a denaturing agent, such as highly concentrated urea and guanidine hydrochloride, or a surfactant. Some of insoluble proteins can be solubilized by the use of these methods. However, there are many proteins which do not dissolve in a physiological salt solution and cannot be solubilized even by using a surfactant, reducing agent, or denaturing agent as described above.

Since immunoassays such as ELISA and affinity electrophoresis need to be conducted in an aqueous solution, both a particular protein (antigen) of interest and an antibody that binds specifically with the antigen and is thus used for measuring the amount of the antigen, must be soluble in the aqueous solution. Therefore, in order to measure an insoluble protein with any of these assays, it is necessary to solubilize the insoluble protein. However, when an insoluble protein is solubilized by a conventional solubilization method, affinity binding sites in the insoluble protein are affected by the used denaturing agent such as urea and the binding activity cannot be maintained. Also, an affinity substance, such as an antibody, having the binding affinity for the insoluble protein is affected by the used surfactant, reducing agent, or denaturing agent, and the binding activity may be decreased by, for example, the conformational change. In addition, when the used solubilizing agents is removed from the assay system so as to restore the binding activity of the affinity substance at the time of the measurement, the protein of interest becomes insoluble again by such removal. Therefore, there was a problem that an insoluble protein could not be measured with immunoassays such as ELISA and affinity electrophoresis because a suitable solubilization method did not exist.

In Western blot method, an antigen is solubilized by a denaturing agent such as a surfactant or a reducing agent, separated with electrophoresis, and transferred into a membrane, and then on the membrane, the antigen is made to be insoluble again by removing the denaturing agent, and the amount is measured using an antibody against the antigen.

It is possible to solubilize an antigen which is insoluble due to disulfide bond or noncovalent bond (such as hydrogen bond, electrostatic interaction, hydrophobic interaction, and van der Waals force) by a denaturing agent added during electrophoresis. However, in the case where an insoluble antigen is a crosslinked collagen or elastin (which is crosslinked by dehydrolysinonorleucine, desmosine, isodesmosine, histidinohydroxymerodesmosine, or the like), a polymerized fibrin (which is crosslinked through isopeptide bond) or the like, the insolubility of all of which is due to covalent bond, there was a problem that it is impossible to measure such an antigen because it is not solubilized by a denaturing agent.

Further, in the case where an antigen is used to produce an affinity substance, such as antibody, that binds specifically with the antigen, there were some problems that the yield of such an affinity substance was low and that heterogeneous affinity substances were produced and thus the purification was required, because of inhomogeneity factors due to the parts other than the affinity binding site in the antigen. There was also a problem that an antibody was not able to be produced against an insoluble antigen.

And further, even when the protein of interest is soluble, it is difficult to measure it with high accuracy by using a conventional measurement method, because the conformation of the protein causes uncertainties.

The present invention has been made in view of the above-mentioned problems, and the inconvenience. The present invention is aimed at providing a method for measuring proteins including insoluble and soluble proteins with high accuracy.

### SUMMARY OF THE INVENTION

The present invention provides a method for measuring a particular protein in a sample containing at least one protein, wherein the sample is reacted with a reagent cleaving a peptide bond of the particular protein to generate a soluble peptide fragment to be detected which is determined by a certain primary structure; and contacted with a reagent reacting specifically with the particular soluble peptide fragment, thereby detecting the presence of the particular soluble peptide fragment.

The present invention also provides a device suitable for use in the above-mentioned method, comprising a flow channel where affinity isoelectric focusing electrophoresis is conducted, an anolyte reservoir which is filled with anolyte, and a catholyte reservoir which is filled with catholyte.

The present invention also provides another device suitable for use in said method, comprising a measurement member for measuring the presence or absence and the concentration of the particular peptide fragment with immunoassay coupled with fluid control.

The present invention provides a method for preparing a peptide fragment which is capable of being bound by a substance having an affinity for a particular protein, wherein a protein preparation containing the particular protein is reacted with a reagent cleaving a protein at a site of a certain amino acid or amino acid sequence to generate a soluble peptide fragment which is determined by a certain primary structure; and contacted with a reagent reacting specifically with the particular soluble peptide fragment, thereby collecting the particular soluble peptide fragment.

The present invention provides a method for screening for a biomarker, wherein a sample containing at least one protein is reacted with a reagent cleaving a protein at a site of a certain amino acid or amino acid sequence to generate soluble peptide fragments which is determined by a certain primary structure; and the soluble peptide fragments are screened for the biomarker.

The present invention provides a testing method for measuring in a biological sample the presence of the biomarker determined by the said screening method.

These and other objects of the present application will become more readily apparent from the detailed description provided hereinafter. It should be understood, however, that the detailed description and specific examples, while disclosing the preferred embodiments of the invention, are provided by way of illustration only, since various changes and modifications within the spirit and scope of the invention will become apparent to those skilled in the art.

### BRIEF DESCRIPTION OF THE DRAWINGS

The present invention will become more fully understood from the detailed description provided hereinbelow and the accompanying drawings which are given by way of illustration only, and wherein:

FIG. 1 illustrates a schematic diagram of an insoluble protein.

FIG. 2 illustrates one embodiment of the measurement methods according to the present invention (in which a particular protein is insoluble).

FIG. 3 illustrates the schematic top view of one embodiment of the measuring devices according to the present invention.

FIG. 4 illustrates the schematic top view of another embodiment of the measuring devices according to the present invention.

FIG. 5 indicates a graph showing the fluorescence intensity pattern along the flow channel which was obtained in Example 1. In the graph, there are four peaks corresponding to complexes of mouse prion with fluorescently labeled antibody. The concentrations of protein in the mouse prion protein-containing samples used are: (a) 1.06 μg; (b) 0.35 μg; (c) 0.035 μg. Note that graph (c) is magnified ten times (×10) in the direction of the ordinate axis.

FIG. 6 shows the entire amino acid sequence of mouse prion protein which was the final target for the measurement in Examples.

FIG. 7 shows the amino acid sequences of the peptide fragments that generated from mouse prion protein by degradation with cyanogen bromide.

### DETAILED DESCRIPTION OF THE INVENTION

(Measurement Method)

The present invention is a method for measuring a particular protein in a sample containing at least one protein, wherein the sample is reacted with a reagent cleaving a peptide bond of the particular protein to generate a soluble peptide fragment which is determined by a certain primary structure (first step); and contacted with a reagent reacting specifically with the particular soluble peptide fragment, thereby detecting the presence of the particular soluble peptide fragment (second step).

(The First Step: Degrading a Particular Protein into Peptide Fragments)

In the first step of the said method, the particular protein existing in the sample is degraded into the constituent peptide fragments. Through the degradation, factors interfering with the measurement are excluded including the instability of the conformation of the particular protein, and the variance of regions other than the region corresponding to the soluble peptide fragment to be detected, thereby allowing high accuracy measurement in the latter step.

A protein of interest (a particular protein) in the method according to the present invention is any protein. The term “a protein” as used herein is intended to mean a polypeptide having any biological activity, and preferably a polypeptide that causes a disease, a disorder or any other abnormality in an animal including a human or appears in associate therewith in the body. A particular protein may be soluble or insoluble. A soluble protein is particularly preferably a protein having a higher-order structure which is unstable. Such a soluble protein can be measured by the method of the invention with high accuracy and stability (for instance, with a more precise quantification), which was difficult by a conventional method because of the instability of the higher-order structure and/or uncertain factors.

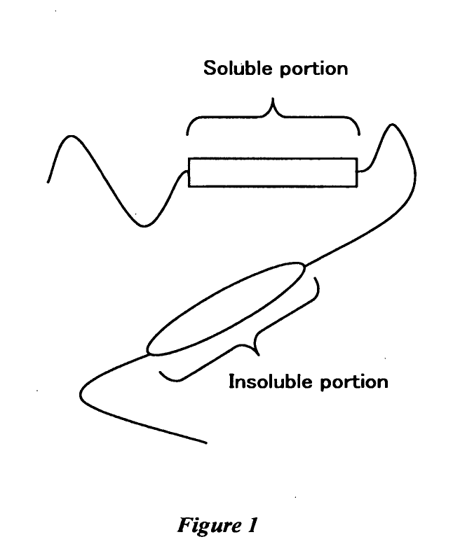

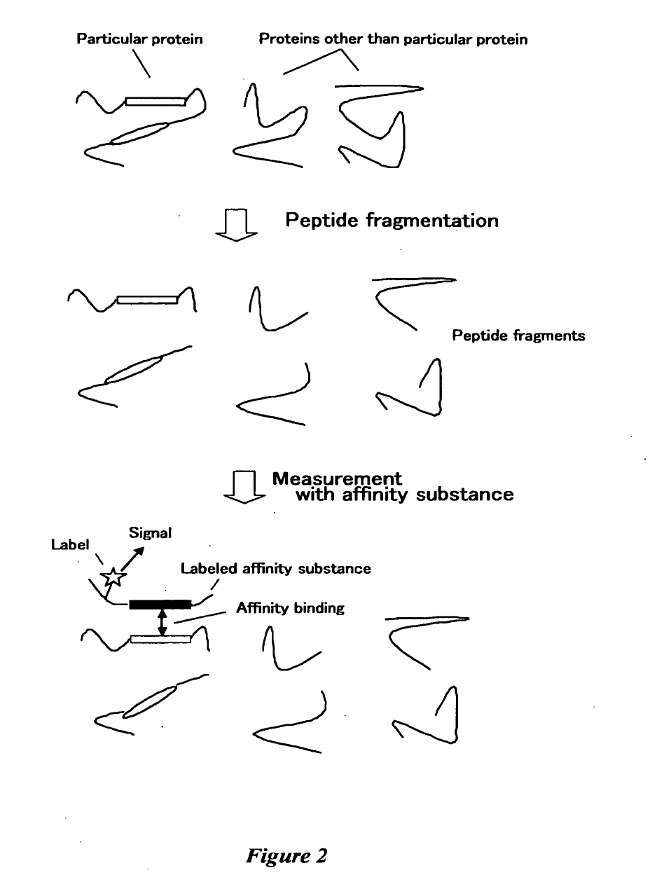

Also, an insoluble protein is particularly preferable for a protein of interest in the method of the invention. Such an insoluble protein can be measured in an aqueous buffer system, in which an insoluble protein per se is difficult to be measured, by the method of the invention though the measurement of soluble, particular peptide fragment generated by degradation (FIGS. 1 and 2). As used herein, the term “insoluble” peptide is intended to mean a peptide that dose not or little dissolve in a physiological salt solution. Physiological salt solutions include, but not limited to, such solutions having a salt concentration of 0.05 to 0.2 M and a pH of 6 to 9, and not containing a solubilizing agent like urea, a surfactant, or a reducing agent, which include, but not limited to, 10 mM sodium phosphate buffer solution containing 150 mM NaCl (pH 7.35), 50 mM tris(hydroxymethyl)aminomethane-HCl buffer solution containing 0.1 M NaCl (pH 7.4), 0.1 M sodium phosphate buffer solution (pH 7.2), 0.1 M 4-(2-hydroxyethyl)piperazine-1-ethanesulfonic acid-NaOH buffer solution (pH 7.4), 0.1 M 3-(N-morpholino)propanesulfonic acid-NaOH buffer solution (pH 7.35), Ringer's solution consisting of 112 mM NaCl, 1.8 mM KCl, 1.1 mM CaCl₂, and 2.4 mM NaHCO₃. Preferable insoluble proteins for use in the present invention are proteins that do not or little dissolve in a physiological salt solution, but that dissolve in a solution containing urea at about 8 M concentration, guanidine hydrochloride at about 6 M concentration, sodium dodecyl sulfate at about 2% (w/v) concentration, and mercaptoethanol at about 5% (w/v) concentration, each alone or in any combination. Examples of this type of proteins include many denatured proteins (thermally denatured albumen, milk protein, and the like), an inclusion body observed when a recombinant protein is expressed in Escherichia coli, many membrane proteins, abnormal prion, amyloid protein, and the like. Also, preferable insoluble proteins for use in the invention are such proteins that do not or little dissolve in both a physiological salt solution and a solution containing urea at about 8 M concentration, guanidine hydrochloride at about 6 M concentration, sodium dodecyl sulfate at about 2% (w/v) concentration, and mercaptoethanol at about 5% (w/v) concentration, each alone or in any combination. Examples of this type of proteins include insoluble proteins in whose insolubility the formation of ε-(γ-glutamyl)lysine isopeptide bond by transglutaminase is involved, and specifically a fibrin gel (the one crosslinked by transglutaminase (blood coagulation factor XIII)), an α2-antitrypsin-fibrin complex, an acquired enamel pellicle (the thin film of glycoprotein formed on the surface of healthy teeth), multimeric fibronectin, lens proteins from cataract patients, scalelike epithelial cells that forms the protective thickened layer of skin (keratinocyte transglutaminase, and epidermal transglutaminase are involved in), insoluble neurofilament, the noncollagenous microfibril in the extracellular matrix (fine fibers), and the coat protein of an eelworm. Proteins showing the same dissolution characteristics (for example, solubility in the physiological salt solution or in both a physiological salt solution and the above-mentioned solution) as that of the proteins illustrated above are preferable for use in the present invention, even if they are proteins other than those as illustrated above.

The sample is any sample which has the possibility of containing a particular protein. The sample may be a biological sample from a subject (a human and an animal including a domestic animal such as cattle, a horse, a pig, a goat, a sheep, a chicken, a rat, and a mouse). Biological samples include, for example, body fluids such as blood (including serum and plasma), lymph, the spinal fluid, ascites, tissue exudate or secretion, phlegm, and urine; tissues (and homogenate, lysate, or extract thereof such as the brain, the spinal cord, the heart, the liver, and the mucous membrane; and cells (and lysate or extract thereof). The sample may be a food sample which is suspected of bacterial infection.

A soluble peptide fragment to be detected (also herein referred to as simply “particular peptide fragment”) can be generated by the degradation of a particular protein and consequently consists of a subsequence (or a partial sequence) of the amino acid sequence of the particular protein. The soluble peptide fragment to be detected has at least one unique part of the particular protein, and the unique part is bound by an affinity substance. It may be determined whether the part, to which an affinity substance binds, of the particular peptide fragment is unique to the particular protein by any method known in the art. For example, it may be determined by conducting a sequence comparison (using, e.g., BLAST or FASTA) of the amino acid sequence of the part with the sequences in the amino acid sequence database (e.g., PRI, UniProt, and NR-AA).

It is known whether the peptide fragment to be detected is soluble or insoluble by subjecting the soluble fraction generated from a particular protein after the treatment with any degrading reagent to mass spectrometric analysis.

When a known antibody is used as an affinity substance, one may use a degradation method to generate a soluble peptide fragment to which the antibody binds. When the genetic information or amino acid sequence information of the particular protein is available, one may use a method to generate a soluble peptide fragment that could be an antigen, based on the information. Alternatively, one may degrade a particular protein by some degradation methods, and produce and/or select some antibodies which bind to the some degradation products respectively.

A particular peptide fragment is not the only one for a particular protein and an affinity substance. It may be of any length as long as it has a site to which the affinity substance binds and is actually bound by the substance. When an affinity substance is one that recognizes a certain amino acid sequence, a particular peptide fragment can consists of contiguous amino acid residues of 4 or more, for example, 5 or more, 6 or more, 7 or more, 8 or more, 9 or more, 10 or more, 12 or more, 15 or more, and 20 or more, because at least four amino acid residues are generally required to be specifically bound by an affinity substance.

A particular peptide fragment is soluble. Because a region which is bound by an affinity substance is often hydrophilic (for example, Kyte, J. and Doolittle, R F., J. Mol. Biol., 157(1): 105-32, 1982; Kyte, J. and Doolittle, R F., J. Mol. Biol., 157: 105-132, 1982: Hopp, T P. and Woods, K R., Mol. Immunol., 20(4): 483-9, 1983) and thus soluble, a peptide consisting of this region may be a particular peptide. fragment. The solubility of a peptide fragment can be evaluated from its amino acid sequence based on, for example, the hydrophobicity index (for example, Kyte and Doolittle, 1982) or the hydrophilicity index (for example, Hopp, T P. and Woods, K R., Mol. Immunol., 20(4): 483-9, 1983). In addition, a region which is bound by an affinity substance can be predicted according to any one of the techniques known in the art, such as Emini, E A., Hughes, J V., Perlow, D S., and Boger, J., J. Virol. 1985 September; 55(3): 836-9.

A particular peptide fragment is not necessary to have a particular conformation, and is determined by a certain primary structure based on the amino acid sequence of the particular protein. The number of amino acids constituting a particular peptide fragment is not limited, and preferably the particular peptide fragment consists of contiguous amino acid residues of, for example, 200 or less, 150 or less, 120 or less, 100 or less, 80 or less, 50 or less, 30 or less, 20 or less, and 15 or less. A soluble peptide fragment to be detected can be specified “only” by a particular primary structure.

The degradation is carried out with an agent that can degrade a protein to generate a soluble peptide fragment to be detected that is determined by a certain primary structure. In the degradation, such an agent may be used that can degrade a protein at a site of a certain amino acid or amino acid sequence (a certain primary structure). An example of this agent includes an enzymatic reaction regent with high substrate specificity. An enzymatic reaction reagent may be, for example, a protease. For example, the following protease can be used for the limited hydrolysis: trypsin for the hydrolysis of the peptide bond on the C terminal side of lysine or arginine; chymotrypsin for the peptide bond on the C terminal side of phenylalanine, tryptophan, or tyrosine; pepsin for the peptide bond on the C terminal side of leucine or phenylalanine; bromelain for the peptide bond on the C terminal side of alanine, lysine, and tyrosine; elastase for the peptide bond on the C terminal side of alanine or glycine; clostripain for the peptide bond on the C terminal side of arginine; V8-protease for the peptide bond on the C terminal side of glutamic acid or aspartic acid; thermolysin for the peptide bond on the N terminal side of leucine or phenylalanine; lysyl endopeptidase for the peptide bond on the C terminal side of lysine; arginine endopeptidase for the peptide bond on the C terminal side of arginine; prolyl endopeptidase for the peptide bond on the C terminal side of proline; aspartic acid-N protease for the peptide bond on the N terminal side of aspartic acid.

For a method for degrading a particular protein into peptide fragments, one can utilize a chemical reaction in which a chemical reagent reacts specifically with a site of amino acid or the amino acid sequence. Examples of chemical reagents for a specific chemical reaction include cyanogens bromide, which cleaves the peptide bond in the C terminal side of methionine; N-bromosuccinimide, BNPS-skatole (3-bromo-3-methyl-2-(2-nitrophenylmercapto)-3H-indole) in 50% acetic acid, dimethyl sulfoxide-HCl-HBr, iodosylbenzoic acid or N-chlorosuccinimide, which cleave the peptide bond in the C terminal side of tryptophan; hydroxylamine, which cleaves the asparagine-glycine bond; and 10% acetic acid containing 7 M guanidine hydrochloride, which cleaves aspartic acid-proline bond.

A regent for degrading a protein into peptide fragments may be a single reagent or a combination of two or more reagents. A combination of reagents may be a combination of enzymatic reaction reagents, a combination of chemical reaction reagents, or a combination of an enzymatic reaction reagent and a chemical reaction reagent. Also, a combination of an enzymatic reaction reagent and/or a chemical reaction reagent with a specific degrading reagent can be used.

A reagent or a combination of reagents used in the first step is selected so that it degrades a particular protein to generate a particular peptide fragment. Since it is unambiguously determined which position(s) of peptide bond is cleaved by a reagent or a combination of reagents which degrades a protein at a site of a certain amino acid or amino acid sequence, one can easily select a reagent or a combination of reagents for degrading a particular protein into a particular peptide fragment based on the amino acid sequence information. A reagent or a combination of reagents which generate a certain particular peptide fragment is not only a single reagent or a single combination. The same particular peptide fragment can also be generated with the use of different reagents or different combinations of reagents. As for a combination of reagents, it is preferable to apply the reagents to a sample sequentially. If they do not interfere with each other in their degrading action, they may be applied at the same time. If they interfere with each other, each of them may be removed or inactivated after its reaction. In the method according to the present invention, unlike a conventional solubilization method, a particular peptide fragment remains soluble after the used reagent is removed.

The first step, in which the soluble peptide fragment to be detected is generated from a particular protein, may be carried out by bringing a reagent or a combination thereof that cleaves a peptide bond at a site of a certain amino acid or a certain amino acid sequence into a contact with the particular protein under such conditions that allow the reagent or the combination to cleave substantially all of the cleavable sites (the peptide bond) of the particular protein. The conditions include, but not limited to, the selection of optimal pH and reaction temperature (for example, 20, 25, 30, 35, 40, 45, 50° C. or more) for the reagent used, a long enough contact (reaction) time (for example, 5 min. or more, 10 min. or more, 15 min. or more, 20 min. or more, 25 min. or more, 30 min. or more, 35 min. or more, 40 min. or more, 45 min. or more, 50 min. or more, 55 min. or more, or 60 min. or more), an enough amount (preferably in 1:1 weight or molar ratio, or excess) of the reagent for the proteins contained in the sample used, and the like.

A reagent that degrades a particular protein into peptide fragments usually acts on a protein in general. Thus, in the case where an affinity substance is a protein such as an antibody, if the reagent remains present in the measurement system at the time of the measurement in the second step, such a affinity substance is also degraded by the reagent, thereby decreasing the affinity binding ability. Therefore, it is desirable to inactivate the reagent or remove it from the measurement system prior to adding the affinity substance into the system.

In the case where the reagent is an enzymatic reaction reagent, it is possible to inactivate the enzyme by adding an inhibitor (for example, a protease inhibitor) that inhibits the activity of the reagent used, or by denaturing the reagent with heating, acid or the like. In the case where the reagent is a chemical reaction reagent, one may use a reagent that inactivates the chemical reaction reagent, or carry out a simple treatment, such as centrifugation, distillation, or solid phase extraction, to remove the reagent. The inactivation or removal of the reagent is preferably carried out by a method specific for the reagent, for example, a method using a specific inhibitor.

Among peptide fragments generated by degradation of a protein, those other than the particular peptide fragment may be soluble or insoluble. The insoluble peptide fragments may be removed from the measurement system by a treatment such as centrifugation so as to prevent from disturbing <disturbance of, troubles of > the measurement system, such as clogging in the flow channel, caused by insoluble substances and to improve the reproducibility of measurements.

(The Second Step: the Measurement of a Particular Peptide Fragment with an Affinity Substance)

In the second step of the method according to the present invention, the peptide fragment to be detected among the peptide fragments generated in the first step is contacted with a reagent specifically reacting therewith thereby detecting the presence of the soluble peptide fragment to be detected. Because there is a proportional relationship between the concentrations of a particular protein to be eventually detected and of a particular peptide fragment, the amount of the particular peptide fragment is proportional to the amount of the particular protein present in the original sample. Especially, if there is a direct proportional relationship between the concentrations of the particular protein and of the particular peptide fragment, the amount of the particular peptide fragment represents the amount of the particular protein in the original sample.

A reagent that reacts specifically with the peptide fragment to be detected (also herein referred to as simply “an affinity substance”) is a substance that has an affinity for a particular peptide fragment and forms a complex therewith in their coexistence. It may be, but is not limited to, a protein, a peptide, a nucleic acid, and a synthesized chemical substance. In the second step, the presence of the particular peptide fragment in a sample is detected by taking advantage of specific affinity binding between a particular peptide fragment and an affinity substance. Therefore, it is necessary that the affinity substance and the particular peptide fragment are both stably present while maintaining the binding activity.

An affinity substance may be, for example, an antibody or a fragment thereof which contains the antigen binding site (including a single chain antibody, an Fab fragment, an F(ab′)₂ fragment, and an Fab′ fragment). An antibody may be a known antibody, or a fragment thereof which contains the antigen binding site. It may be an antigen that is newly produced for use in the measurement method according to the present invention, or a fragment thereof which contains the antigen binding site. Preferably, the amino acid sequence of the site in the particular peptide fragment, to which an antibody or its binding fragment binds, is known. An antibody or a fragment thereof which contains the antigen binding site is preferable to be monoclonal. A method for producing an antibody to a peptide is known in the art. Briefly, such an antibody can be obtained from the serum of an animal (for example, an animal which can be used for producing an antibody including a mouse, a rat, a rabbit, a hamster, a guinea pig, a goat, a sheep, and a chicken) by immunizing the animal once or more with a peptide alone, or together with an adjuvant (for example, a complete or incomplete Freund adjuvant, aluminum hydroxide or aluminum phosphate (alum)), or in conjugation with an appropriate carrier (for example, albumin like BSA, ovalbumin, keyhole limpet hemocyanin, diphtheria toxin, and tetanus toxin). A method for producing a monoclonal antibody is well-known in the art (see, for example, Köhler and Milstein (Nature, 256: 495-497, 1975; Antibodies: A Laboratory Manual, ed. Harlow and Lane, Cold Spring Harbor Laboratory, 1988). Briefly, a monoclonal antibody can be obtained by the following: an animal is immunized with the peptide; the spleen cells (B cells) are isolated from the animal and then fused to myeloma cells of the same or a closely-related species by the cell fusion technique to obtain immortal cell lines (hybridomas); the hybridomas are grown and finally screened for the production of antibody capable of binding to the peptide. An antibody may be, but not necessary, capable of binding to the original particular protein. In the method according to the present invention, an antibody is only required to have the ability of binding to the particular peptide fragment.

If a particular peptide fragment contains a sugar chain, an affinity substance may be, for example, a protein, such as lectin, which recognizes the sugar chain bound to a protein.

An affinity substance may be a nucleic acid ligand which binds specifically to a particular peptide, such as aptamer. Aptamer is DNA or RNA which has the base sequence and the structure capable of recognizing a particular peptide. For an affinity substance of nucleic acid having the binding affinity for the particular peptide fragment derived from a particular protein, one selects the nucleic acid having the affinity binding for the particular peptide fragment from among a population of nucleic acids having random base sequences by the use of a screening method such as, for example, the SELEX method (in vitro selection).

Affinity substances of synthetic chemicals include high molecular substances. For an affinity substances of this type, high molecular substances synthesized by, for example, molecular imprinting are screened for the conformation recognizing the particular peptide fragment and the presence of an affinity functional group which causes, for example, the electrostatic interaction with the particular peptide fragment.

An affinity substance preferably has a label for measurement. Labels used for measurement are known in the art. Labels are measured optically, chemically, radioactively, magnetically, or electrically, and preferably measured optically or electrically. A label is selected from the group consisting of, for example, fluorescent dyes, enzymes, absorbing pigments, chemiluminophores, radioisotopes, spin labels, and electrochemical labels.

For the measurement with higher accuracy, a single particular peptide fragment may be measured with two or more affinity substances. Examples of measurements of this type include, for example, the sandwich technique. Two or more of particular peptide fragments derived from a single particular protein may be measured with the use of two or more respective affinity substances.

A method for measuring the existence of a particular peptide fragment using an affinity substance can be any of the known methods that utilize affinity binding between two substances. Preferably, it is a method utilizing affinity binding between a peptide fragment and a protein, a peptide, a nucleic acid, or a synthesized chemical.

When an antibody or a fragment thereof containing the antigen binding site is used as an affinity substance, an immunoassay can be used for the measurement. An immunoassay is a specific measurement method utilizing a specific binding reaction between an antigen and an antibody, which is known in the art. Even if a substance (for example, a nucleic acid or a synthesized chemical) other than an antibody is used as an affinity substance, the specific affinity binding reaction between an affinity substance and a particular peptide fragment is used for the measurement. Therefore, those skilled in the art could easily understand that such a method is the same in principle as an immunoassay and one can select or design an appropriate measurement depending on the affinity substance used. Thus, it should be noted that in the present specification, the term “immunoassay” is meant to also include any methods taking advantage of any other affinity binding reactions than antigen-antibody reaction, except for the case where it clearly refers to only the method utilizing the antigen-antibody reaction.

A measurement method preferably measures the presence/absence and the amount of the binding reaction between a particular peptide fragment and an affinity substance, in order to know the presence/absence and the amount, if any, of a particular protein to be eventually detected. A quantitative measurement can be achieved by measuring, for example, UV, fluorescence, radioactivity, magnetism, electrical conductivity or the like which is due to the label.

For the measurement with higher accuracy and more specificity, one may measure the amino acid sequence of the particular peptide fragment bound by an affinity substance with, for example, mass spectrometry (MS), or measure a combination of affinity binding and any of the physicochemical properties other than affinity binding, such as the isoelectric point and the molecular weight. Also, one can measure the presence/absence (and the amount preferably) of two or more particular peptide fragments derived from a single particular protein to be eventually detected. The measurement of two or more particular peptide fragments make it possible to achieve the measurement with higher accuracy.

Measurements for use in the method according to the present invention include affinity electrophoresis (especially, affinity isoelectric focusing electrophoresis), immunoassay such as ELSA, Western blot method, the SELDI-MS method, which measures a protein bound to an antibody immobilized on a chip with mass spectrometry (MS) (an instrument for this measurement method is commercially available from, for example, Ciphergen Biosystems, Inc., USA), and surface plasmon resonance (SPR) method, which detects the binding between affinity substances as the change in the refractive index by surface plasmon resonance (an instrument for this measurement method is commercially available from, for example, Biacore AB, Sweden). It is particularly preferable to use affinity isoelectric focusing electrophoresis, which allows a rapid, easy, and automatic measurement with high sensitivity, accuracy and specificity.

Prior to measuring a particular peptide fragment (after the first step and before the second step), one can remove or separate the other peptide fragments (both those derived from a particular protein and those derived from the other proteins) generated by the degradation in the first step with the use of any of the various means such as electrophoresis, liquid chromatography, gel filtration, centrifugal separation, and solid phase extraction. This prevents other peptide fragments than the particular peptide fragment from interfering with the measurement system, thereby achieving the measurement of the particular protein with higher accuracy.

(Measurement Kit)

The measurement kit according to the present invention comprises a reagent (or a combination of reagents) which degrades a particular protein into peptide fragments and an affinity substance having the affinity binding to a particular soluble peptide fragment derived from the particular protein. The affinity substance may be labeled. The present measurement kit is suitable for use in the above-mentioned measurement method according to the present invention. The present kit may further comprise a second reagent deactivating the reagent that degrades a particular protein into peptide fragments. The reagent that degrades a particular protein into peptide fragments, an affinity substance and another reagent deactivating the reagent that degrades a protein into peptide fragments, and the others are the same as those described for the above-mentioned measurement method.

(A Device for use in Affinity Isoelectric Focusing Electrophoresis)

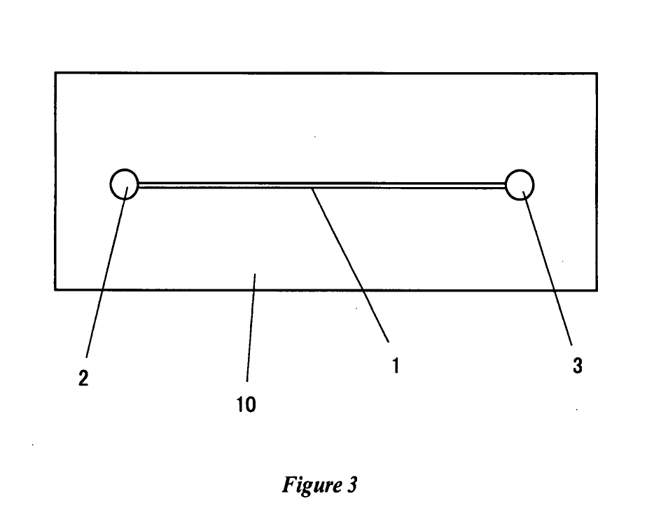

Hereinafter, the device according to the present invention that uses affinity isoelectric focusing electrophoresis will be described referring to the figures. FIG. 3 is the schematic top view of the present measuring device. As shown in FIG. 3, the measuring device according to the present invention comprises substrate 10 on which flow channel 1 for conducting affinity isoelectric focusing electrophoresis, anolyte reservoir 2 which is filled with anolyte, and catholyte reservoir 3 which is filled with catholyte are formed. As the material of substrate 10, for example, plastic materials, glass, quartz, photocuring resins, thermosetting resins, and the like can be used. The cross sectional shape of flow channel 1 formed on substrate 10 is not especially limited but may be, for example, a rectangle, a round shape, or a trapezoid or the like. Also, the flow channel may have a round bottom. Flow channel 1 is not necessarily in a linear shape, but may be in a meander shape, an eddy shape, a spiral shape, or the like. The top view shapes of anolyte reservoir 2 and catholyte reservoir 3 which are formed on substrate 10 may be, for example, circular, elliptical, rectangular, or the like. Anolyte reservoir 2 and catholyte reservoir 3 may exchange their positions. A reservoir which is filled with anolyte is an anolyte reservoir and a reservoir which is filled with catholyte is a catholyte reservoir. It is desirable that flow channel 1, anolyte reservoir 2, and catholyte reservoir 3 are formed by removing the surface of substrate 10 partially in the direction of its thickness by, for example, wet etching, dicing saw, or the like. Also, one can prepare a mold having convex portions corresponding the shapes of flow channel 1, anolyte reservoir 2, and catholyte reservoir 3 in the mold cavity, and perform injection molding, hot embossing, or the like using the mold to manufacture substrate 10 on which flow channel 1, anolyte reservoir 2, and catholyte reservoir 3 are formed. It is not necessary that all of Flow channel 1 and reservoirs 2 and 3 are formed on the same substrate. Substrate 10 having flow channel 1 is formed thereon is bonded to another substrate (not shown) on which two through-holes are prepared and used for respective reservoirs. Moreover, the flow channel and reservoirs need not be formed on a substrate(s). For example, one can use a measuring device wherein a hollow capillary (such as quartz hollow capillary), as a flow channel, is connected to vessels (such as plastic vessels) which can be respectively filled with anolyte and catholyte, as reservoirs.

For isoelectric focusing electrophoresis, flow channel 1 is filled with an aqueous solution containing a plurality of carrier ampholites having both weakly acidic and weakly basic dissociating groups. The range of pH (pI) gradient in the carrier ampholites can be selected so that the isoelectric point of the complex of a particular peptide fragment to be detected with an affinity substance is within the range. Anolyte reservoir 2 is filled with acidic anolyte such as, for example, phosphoric acid solution, and catholyte reservoir 3 is filled with basic catholyte such as, for example, sodium hydroxide solution. In order to prepare a pH gradient in flow channel 1, one may use an immobilized pH gradient gel wherein weakly acidic and weakly basic dissociating groups have previously been immobilized, or polyacrylamide or agarose gel which contains carrier ampholites, instead of an aqueous solution containing carrier ampholites. When immobilized pH gradient gel is used, the anolyte and the catholyte are not necessarily required.

It is preferable that the width of flow channel 1 is, for example, in the range of 1 μm to 5000 μm, and the depth is, for example, in the range of 1 μm to 5000 μm, and the length is, for example, in the range of 0.1 cm to 50 cm, although these size parameters are not limited to the ranges. The smaller width and depth of the flow channel make it possible to apply a high voltage while suppressing the generation of Joule heat during isoelectric focusing electrophoresis, thereby achieving a rapid separation. The surface of the flow channel may be treated with, for example, polydimethylacrylamide or the like so as to prevent the adsorption of peptide fragments and affinity substances or the electroosmotic flow. As for anolyte reservoir 2 and catholyte reservoir 3, it is preferable that, for example, the diameter is in the range of 1 μm to 5000 μm and the depth is in the range of 1 μm to 5000 μm, but the diameter and the depth are not limited to the ranges. The reservoirs each may include an electrode (not shown). For example, the electrode may be formed in the respective reservoirs on the substrate by sputtering. Alternatively, for example, the respective reservoirs may be adapted to have a mechanism for holding inserted electrodes and when needed, the electrodes can be inserted in the mechanisms on the substrate. During electrophoresis, a voltage is applied between the electrodes. The voltage is desirably a direct current voltage. The measuring device according to the present invention may comprise a mechanism for applying a voltage. Substrate 10 on which flow channel 1, anolyte reservoir 2, and catholyte reservoir 3 have been formed may be adapted to be covered by another substrate (not shown). The device according to the present invention may further comprise a detection device to detect a reagent (for example, an affinity substance) bound to a particular peptide fragment. The detection device is preferably a photodetection device. The photodetection device includes a light source and a detector. The light source is preferably selected from the group consisting of lasers, LED, and lamps. The detector is preferably selected from the group consisting of photoelectron multipliers and multipixel photodetectors.

(Isoelectric Point Separation)

The measurement using the affinity isoelectric focusing electrophoresis will be described. An aqueous solution containing a plurality of carrier ampholites having both weakly acidic and weakly basic dissociating groups is admixed with the mixture of a sample after the degradation in the first step in the method according to the present invention and an affinity substance to obtain a sample-loading solution for use in the affinity isoelectric focusing electrophoresis. The admixing may be carried out at the same time of mixing the sample after the degradation with the affinity substance.

Flow channel 1 is introduced with the sample-loading solution. Alternatively, one may introduce the solutions of the sample after degradation, the affinity substance, the carrier ampholite, and the like into flow channel 1 independently, or in premixture of any combination, and in any order. The solution(s) may be introduced into flow channel 1 via reservoir 2 or 3, under pressure, or by taking advantage of capillary action. After flow channel 1 is filled with the sample-loading solution, anolyte reservoir 2 and catholyte reservoir 3 are filled with anolyte and catholyte respectively. Then, a voltage is applied between the electrode of anolyte reservoir 2 as an anode and the electrode of catholyte reservoir 3 as a cathode to conduct isoelectric focusing electrophoresis. The voltage applied is, for example, in the range of 100 to 1000 V per 1 cm of the flow channel length, although it is not limited to this range. During electrophoresis, the measurement system may be cooled by means of, for example, Peltier (not shown) in order to exclude the influence of Joule heating. The voltage application allows pH gradient to be formed along flow channel 1 introduced with the carrier ampholite. A peptide (including a protein) is amphoteric, and the isoelectric points varies with the side chain dissociating groups of the amino acids constituting the peptide, the amino group in N terminal, and the carboxyl group in C terminal. Therefore, the peptides present in the flow channel converge to the position of the pH equal to the respective isoelectric point and thus are separated. The separation by isoelectric focusing electrophoresis is achieved within a time of 0.1 to 10 minutes, but not limited to this range.

In the measurement, one obtains a signal from the label that has been previously conjugated to an affinity substance. For example, when a fluorescent dye is used, the excitation light corresponding to the excitation wavelength of the fluorescent dye is irradiated, and the fluorescence emitting from the fluorescent dye is obtained. A fluorescent dye label does not require such an operation as adding a substrate, which is required for an enzymatic label, after electrophoretic separation. An excitation light source may be a laser, LED, a lamp, or the like, and if required, it is possible to use, for example, a filter such as a band-pass filter so as to irradiate only the light of the excitation wavelength. The excitation light may be irradiated from above or below, or, the right or left of the flow channel. The excitation light may be introduced from one of the ends of the flow channel and conducted along the flow channel as a waveguide. It is desirable that the refractive index of the substrate at the wavelength of incident light is lower that that of the solution filled in the flow channel. When the flow channel is used as a waveguide, the strength of the excitation light can be raised, and noise components, such as the reflected light and scattered light from the surface of the flow channel, to the fluorescent measurement instrument are decreased, as compared with the case where the incident light is irradiated from above or below, or, the right or left of the flow channel. Therefore, use of the flow channel as a waveguide allows for the measurement with higher sensitivity. For obtaining fluorescence, one uses, for example, a photoelectron multiplier or a multipixel photodetector (such as a line CCD camera, an area CCD camera, a line CMOS camera, an area CMOS camera, and the like). It is general to use, for example, a filter such as a band-pass filter or a notch filter so as to measure only the light corresponding to the fluorescence emitted from the used fluorescent dye. It is also possible to use any combination of a light source and a detector other than those mentioned above.

The measurement may be carried on the entire flow channel, or may be carried out only at the position of the isoelectric point of the complex of the particular peptide fragment with the affinity substance. In the case of the measurement of the entire flow channel, for example, the excitation light is irradiated to the entire flow channel and the fluorescent signal is obtained from the entire flow channel by imaging it with the use of a combination of a lamp and a CCD camera, or, the substrate is moved by a moving stage and scanned by the fixed optical system, or the substrate is fixed and scanned by the moving optical system with the use of a laser-a photoelectron multiplier.

Measurement methods for isoelectric point separation carried out in an aqueous solution are known in the art. For example, the measurement is carried out by scanning the entire flow channel while applying a voltage, or by conducting the migration by electroosmosis simultaneously with isoelectric focusing electrophoresis to move an ion of interest to the desired detection point.

As described above, peptides in the flow channel converge to the respective positions of the pH equal to their respective isoelectric points and are separated from each other. Because the position of the isoelectric point of the particular peptide fragment-affinity substance complex can be known in advance by a preliminary experiment, the signal from the desired complex can be distinguished by its position from the other signals, for example, signals caused by unbound affinity substances, affinity substances nonspecifically bound to peptides other than the particular peptide fragment, and affinity substances absorbed to the flow channel and the like. This allows for the measurement of a particular peptide fragment to be detected with high accuracy and specificity by excluding false detection. The amount of the particular peptide fragment can be calculated based on the amount of the signal from the label at the position of the isoelectric point of the complex. Because the amount of the particular peptide fragment generated by the peptide fragmentation is proportional, and preferably equal, to the amount of the particular protein before the fragmentation, the amount of the particular protein before the peptide fragmentation can be calculated by measuring the amount of the particular peptide fragment.

The followings are also the advantages of the measurement by affinity isoelectric focusing electrophoresis after peptide fragmentation.

Because the complex of interest converges to its isoelectric point and is thus concentrated, it is possible to detect the complex even if it is in such a minute amount as being dispersed and hidden in background noise in other electrophoretic modes.

When the sample contains a high concentration protein which is not a particular protein to be detected, a pretreatment is generally required to selectively remove the high-concentration protein because it is converged to the isoelectric point by isoelectric focusing electrophoresis and precipitated, thereby affecting isoelectric focusing electrophoresis adversely. However, in the measurement method according to the present invention in which the protein is measured after peptide fragmentation, it is possible to measure the particular protein without the need for such a pretreatment as described above, because the high concentration protein is degraded into a plurality of the peptide fragments which have different isoelectric points, thereby preventing the convergence to a single isoelectric point and the precipitation of the macromolecule.

Because the isoelectric point of the complex varies with or without the posttranslational modification of a particular protein to be detected, it is possible to measure differentially the particular protein with and without the posttranslational modification.

Because neither the peptide fragment to be detected nor the labeled affinity substance is immobilized on the surface of the substrate or the like, the peptide fragment to be detected and the affinity substance react to form the complex freely in solution with high efficiency. Therefore, none of immobilization operation and washing operation for removal of unreacted affinity substances is required.

An affinity isoelectric focusing electrophoresis allows for the measurement with high accuracy by using a single affinity substance, and also allows for the measurement of low molecular weight proteins and peptides.

In the present invention, the complex of the particular peptide fragment and the labeled affinity substance may be measured by taking advantage of UV absorbance or electric conductivity of the complex at the isoelectric point, in place of the light measurement using fluorescent dye. Additionally, it is possible to use all the methods which can recognize the information on the complex.

(A Device for use in Immunoassay Coupled with Fluid Control)

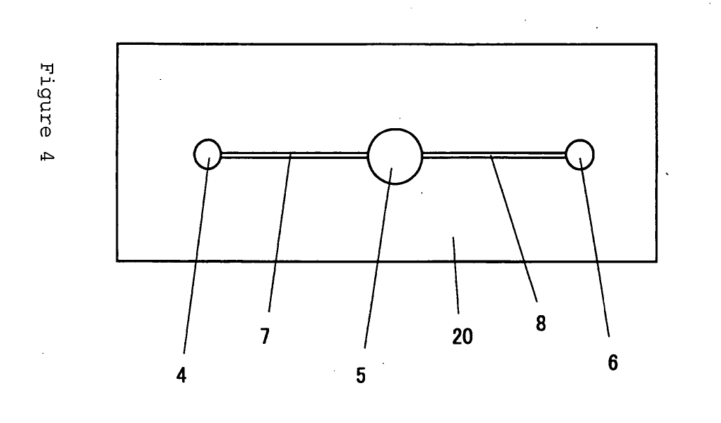

Hereinafter, the device according to the present invention that uses an immunoassay coupled with fluid control will be described. FIG. 4 represents the schematic top view of the measuring device according to the present invention. As shown in FIG. 4, the measuring device according to the present invention comprises substrate 20 on which introduction member 4 for introducing a sample and other, measurement member 5 for measuring a particular peptide derived from a particular protein by recognizing the peptide with affinity binding, drainage member 6 for draining waste fluid, flow channel 7 for connecting the introduction member with the measurement member, and flow channel 8 for connecting the measurement member with the drainage member are formed. Introduction member 4 and drainage member 6 are not necessarily formed, and in the case where they are not formed, neither flow channel 7 nor flow channel 8, which connect the measurement member to the introduction member and the drainage member, respectively, are formed. As the material of substrate 20, for example, plastic materials, glass, quartz, photocuring resins, thermosetting resins, and the like can be used. The top view shapes of introduction member 4, measurement member 5, and drainage member 6 formed on substrate 20 are not especially limited but may be, for example, circular, elliptical, rectangular, or the like. The bottom may be flat or rounded. The cross sectional shape of flow channels 7 and 8 may independently be a rectangle, a round shape, or a trapezoid or the like. Also, the flow channels may independently have a round bottom. Flow channel 7 and 8 are not necessarily in a linear shape, but may independently be in a meander shape, an eddy shape, a spiral shape or the like. Introduction member 4, measurement member 5, drainage member 6, and flow channels 7 and 8 may be formed by removing the surface of substrate 020 partially in the direction of its thickness by, for example, wet etching, dicing saw, or the like. Also, one can prepare a mold having convex portions corresponding the shapes of introduction member 4, measurement member 5, drainage member 6, and flow channels 7 and 8 in the mold cavity, and perform injection molding, hot embossing, or the like using the mold to manufacture substrate 20 on which introduction member 4, measurement member 5, drainage member 6, and flow channels 7 and 8 are formed. It is not necessary that all of introduction member 4, measurement member 5, drainage member 6, and flow channels 7 and 8 are formed on the same substrate. Substrate having measurement member 5 and flow channels 7 and 8 are formed thereon is bonded to another substrate (not shown) on which two through-holes are prepared and used for the introduction member and the drainage member, respectively. Moreover, introduction member 4, measurement member 5, drainage member 6, and flow channels 7 and 8 need not be formed on a substrate(s). For example, one can use a plastic vessel such as a microtitre plate as the measurement member. In this case, the introduction member, the drainage member, and the flow channel are not necessarily required.

It is preferable that the diameter of measurement member 5 is, for example, in the range of 1 μm to 10,000 μm, and the depth is, for example, in the range of 1 μm to 10,000 μm, although these size parameters are not limited to the ranges. In measurement member 5, an affinity substance that recognizes and binds to a particular peptide derived from a particular protein of interest, or a known concentration of a particular peptide derived from a particular protein is introduced before the measurement. The introduced affinity substance or particular peptide of known concentration may be immobilized on measurement member 5 of the substrate. The immobilization methods include, for example, adsorption using hydrophilicity or hydrophobicity, or covalent bonding between each of the materials and the substrate. It is not necessary that the materials are immobilized directly to the substrate. For example, each substance may be immobilized on beads, and then the beads may be introduced in measurement member 5. In order to prevent other proteins and peptide fragments from adsorbing to the measurement member, the measurement member may be blocked. As a material for blocking, for example, bovine serum albumin (BSA) and the like can be used.

It is preferable that the diameter of introduction member 4 and drainage member 6 is, for example, in the range of 1 μm to 10,000 μm and the depth is, for example, in the range of 1 μm to 10,000 μm, although these size parameters are not limited to the ranges. It is preferable that the width of flow channels 7 and 8 is, for example, in the range of 1 μm to 5,000 μm and the depth is, for example, in the range of 1 μm to 5,000 μm, although these size parameters are not limited to the ranges. For introducing a sample and reagents for the measurement, a tube (not shown) may connect introduction member 4 to a solution feeding system (for example, a solution feeding pump such as a syringe pump and a peristaltic pump; not shown), and sample introduction and solution sending may be carried out by the solution feeding system. For draining peptide fragments other than the peptide fragment of a particular protein and reagents for the measurement, another tube (not shown) may connect drainage member 6 to a solution feeding system (for example, a solution feeding pump such as a syringe pump and a peristaltic pump; not shown), and waste fluid may be drained from drainage member 6 on the substrate outside of the substrate by the solution feeding system. If the measuring device consists only of the measurement member, the measurement member may be directly connected to a tube, and the introduction and drainage of a sample and reagents may be carried out with a pump. Instead of pump is used, pipetting or the capillary action can be used for introduction and/or draining various solutions. The measuring device, except for the introduction member, or the introduction member and the drainage member, may be adapted to be covered by another substrate. In order that a particular peptide derived from a particular protein efficiently reacts with an affinity substance, the measurement member may have a stirring mechanism or the like.

The present device may further comprise a detection device to detect a reagent (for example, an affinity substance) which has been bound to a particular peptide fragment. The detection device is preferably a photodetection device. The photodetection device comprises a light source and a detector. The light source is preferably selected from the group consisting of lasers, LEDs, and/or lamps. The detector is preferably selected from the group consisting of photoelectron multipliers and multipixel photodetectors.

(Immunoassay Coupled with Fluid Control)

The measurements with immunoassay coupled fluid control will be described.

Prior to the measurement, a first unlabeled affinity substance or a known concentration of an unlabeled peptide (consisting of the same amino acid sequence as that of a particular peptide fragment) is introduced into measurement member 5. These substances may be immobilized on measurement member 5 of the substrate. The immobilization methods include, for example, adsorption, covalent bonding, and the like. These substances may be immobilized on beads or the like, and then introduced in measurement member 5. The immobilization makes it possible to prevent the substances from moving from measurement member 5 to drainage member 6 by solution feeding and/or washing. In order to prevent other proteins and peptide fragments from adsorbing to measurement member 5 and to achieve the measurement with high accuracy, the measurement member may be blocked. As a material for blocking, for example, bovine serum albumin (BSA) and the like can be used.

(1. The Measurement with an Affinity Substance being Immobilized to the Measurement Member)

After a first unlabeled affinity substance is immobilized on measurement member 5, the sample subject to the degradation in the first step of the method according to the present invention is introduced into measurement member 5 through introduction member 4. The sample may be introduced directly into measurement member 5, not through introduction member 4. As a solution used for introduction and solution feeding, for example, a buffer solution can be used. When the sample arrives in measurement member 5, a particular peptide fragment in the sample reacts to bind specifically with the first affinity substance in measurement member 5. When the first affinity substance is immobilized on measurement member 5, only the particular peptide fragment is immobilized to measurement member 5 via the first affinity substance by this specific binding. After introduction, for example, stirring may be carried out so that the particular peptide fragment reacts efficiently to bind with the first affinity substance. Next, peptides other than the particular peptide fragment are washed out from measurement member 5 to drainage member 6 by feeding a solution to measurement member 5 directly or through introduction member 4. The washing may be repeated several times. Then, a second affinity substance, which recognizes a site different from the site recognized by the first affinity substance in the particular peptide fragment, is introduced into measurement member directly or through introduction member 4. The second affinity substance may be labeled or unlabeled. As a label, for example, fluorescent dyes, enzymes, chemiluminophores, absorbing pigments, radioisotopes, spin labels, and electrochemical labels can be used. If the second affinity substance is not labeled, one can use any of the measurement method that does not need to use a label, including a method for measuring UV absorption. When being introduced into measurement member 5, the second affinity substance reacts to bind specifically with the particular peptide fragment that has been bound to the first affinity substance in measurement member 5. At this time, for example, stirring may be done so that the particular peptide fragment reacts efficiently to bind with the second affinity substance. It is desirable that the second affinity substance exists in excess with respect to the particular peptide fragment. Next, the second affinity substance not bound to the particular peptide fragment is washed out from measurement member 5 to drainage member 6 by feeding a solution to measurement member 5 directly or through introduction member 4. The washing may be repeated several times. As the result of the above-mentioned operations, in measurement member 5, the first affinity substance and the second affinity substance are bound to the particular peptide fragment in a sandwich manner. Because the concentration of the particular peptide fragment and the concentration of the second affinity substance are equal or correlated with each other, the amount of the particular peptide fragment can be determined by measuring the concentration of the second affinity substance.

For the measurement, the same measurement methods as the above-mentioned methods, by which the soluble peptide fragment to be detected is measured with affinity electrophoresis, can be used for the measurement. Also, for example, the SPR method and the SELDI method can be used, in which a single affinity substance is immobilized to the measurement member for the measurement. By using two affinity substances which each recognize two different affinity binding sites in a particular peptide fragment, a particular protein can be measured with high accuracy and specificity.

(2. The Measurement with a Known Concentration of a Peptide being Immobilized to the Measurement Member)

After a known concentration of an unlabeled peptide (which consists of the same amino acid sequence as the particular peptide fragment) is immobilized on measurement member 5, the mixture of the sample subjected to the degradation in the first step of the method according to the present invention and a known concentration of an affinity substance is introduced into measurement member 5 through introduction member 4. For immobilizing the peptide consisting of the same amino acid sequence as the particular peptide fragment on measurement member 5, for example, one can use covalent binding or adsorption. After immobilizing, the measurement member may be blocked. The particular peptide fragment in the sample and the affinity substance have formed a complex by mixing before introduction. It is desirable that the affinity substance exists in excess with respect to the particular peptide fragment in the sample. The mixture may be directly introduced into measurement member 5, not through introduction member 4. As a solution used for introduction and solution feeding, for example, a buffer solution can be used. In measurement member 5, excess affinity substance not forming the complex reacts to bind specifically with the known concentration of the peptide immobilized previously on measurement member 5. The affinity substance forming the complex at the time of introduction does not react with the peptide immobilized previously on measurement member 5. It is desirable that the concentration of the peptide immobilized on measurement member 5 is equal to, or higher than, the concentration of the affinity substance before being introduced. After introduction for example, stirring may be done so that the unbound affinity substance reacts efficiently to bind with the peptide immobilized on the measurement member. Next, the affinity substance not forming a complex with the peptide immobilized on measurement member 5 (and also the particular peptide fragment degraded from the particular protein in the sample) is washed out from measurement member 5 to drainage member 6 by feeding a solution to measurement member 5 directly or through introduction member 4. The washing may be repeated several times.

As the result of the above-mentioned operations, in measurement member 5, the affinity substance which has not formed the complex at the time of introduction is bound to the known concentration of the peptide (consisting of the same amino acid sequence as the particular peptide fragment) that has been immobilized on the measurement member. If no particular peptide fragment exists in the sample, all amount of the affinity substance added in the sample reacts to bind with the known concentration of the peptide immobilized on measurement member 5. If the particular peptide fragment exists in the sample, the amount of the affinity substance to bind with the known concentration of the particular peptide fragment immobilized on measurement member 5 is decreased. Therefore, the presence of the particular peptide fragment in the sample can be confirmed by measuring the amount of the affinity substance which remains in measurement member 5 after washing. Moreover, the amount of the particular peptide fragment present in the sample can be determined based on the amount of the affinity substance remaining in measurement member 5, because they are correlated with each other.

For the measurement of the complex, the same measurement methods as the above-mentioned methods by which the soluble peptide fragment to be detected is measured with affinity electrophoresis can be used for the measurement. Because the measurement with affinity electrophoresis requires that the particular peptide fragment is bound by a single affinity substance, it is possible to measure the particular peptide fragment which is shorter.

In the above-mentioned embodiments, the methods of measuring a particular protein, in which the particular protein is fragmented into peptides and a particular peptide fragment is measured by means of an affinity substance binding thereto specifically, have been described in the case where it is combined with immunoassay coupled with fluid control. However, it should be noted that the measurement method and measuring device according to the present invention can be used by being coupled with any of the techniques or situations in which substances are separated, including immunoassay such as Western blotting method, chromatography, mass spectrometry, and the like.

(Method for Preparing a Particular Peptide Fragment Capable of Binding to an Affinity Substance from a Particular Protein)

It is possible to preparing a particular peptide, to which an affinity substance binds, from a particular protein by using the above-mentioned method of protein fragmentation according to the present invention. Briefly, a protein sample containing a particular protein is subjected to the fragmentation as described above so that all of the proteins contained in the sample degraded into peptide fragments; from among them a particular peptide fragment derived from the particular protein is purified by a means of separation such as immunochemical separation, chromatography, electrophoresis, gel filtration, centrifugation, solid phase extraction, or the like. In addition, after obtaining the particular peptide fragment and determine its amino acid sequence once, the particular peptide fragment may be prepared by peptide synthesis methods (chemical synthesis, genetic recombination, and the like).

According to provision of a particular peptide derived from a particular protein, it is possible to conduct a highly accurate experiment, research, and development under wider range of conditions for storage and experiment. Moreover, because a particular peptide derived from a particular protein has a few inhomogeneity factors caused by the parts other than those corresponding to the particular peptide, and because an insoluble protein can be analyzed that has been difficult to be analyzed in itself by conventional methods, the particular peptide fragment contributes to medical science, drug development, agriculture, and the like.

Further, a particular peptide derived from a particular protein can also be used for preparing an affinity substance for the particular protein. Because the particular peptide fragment prepared by the preparing method according to the present invention has a few inhomogeneity factors due to the parts other than the affinity binding site contained therein, a particular affinity substance can be prepared with high accuracy. Moreover, because a particular peptide derived from a particular protein does not have an unstable conformation factor, it is possible to set wider range of conditions for storage and experiment, and also it is possible to prepare an affinity substance for the particular peptide fragment with good reproducibility and high yield. As an affinity substance, all substances having affinity binding such as a protein, a peptide, nucleic acid, and a synthetic chemical can be prepared. The method of preparing an antibody protein as an affinity substance include, for example: a particular peptide derived from a particular protein is injected as an immunogen into an animal such as a mouse, a rat, or a chicken or into a plant; and the antibody is produced in the body of the animal or the plant. The method for preparing an affinity substance such as a peptide, nucleic acid, or a synthetic chemical include, for example: an affinity substance having high binding affinity for a particular peptide derived from a particular protein is screened. Moreover, because a particular peptide derived from a particular protein has a lower molecular weight than a protein has, an affinity substance that binds specifically to the amino acid sequence of the particular peptide can be synthesized based on its structural information obtained by simulation or the like. The preparing methods are not limited to those as described above, and include all of the methods which prepare an affinity substance using a particular peptide which has been prepared from a particular protein by the method of fragmenting a protein according to the present invention.

(Screening Method for a Biomarker and Testing Method using a Biomarker)

It is possible to screen for a biomarker which can be used for diagnosing a disease or assessing an environment from among the proteins contained in a sample by using the protein-fragmenting method according to the present invention, as described above. The screening method includes, for example, a protein sample containing a plurality of proteins is subjected to the fragmentation as described above so that all of the proteins contained in the sample degraded into peptide fragments; then the peptide fragments are screened for a particular peptide fragment derived from the particular protein with the use of a technique such as immunoassay, chromatography, electrophoresis, mass spectrometry, gel filtration, centrifugation, preferably immunoassay. In the screening method or testing method according to the present invention, a preferable immunoassay is an affinity electrophoresis (for example, affinity isoelectric focusing electrophoresis) or an immunoassay coupled with fluid control.

A biomarker for, e.g., a particular disease may be any peptide that has a difference in its content, electrical properties, molecular weight, or the like, as compared between in a sample from a subject suffering the particular disease and in a sample from a healthy subject. A biomarker is not limited to a peptide capable of being bound by an affinity substance. A biomarker may be a peptide whose capability to bind to an affinity substance is different in between a sample from a healthy subject and a sample from a subject suffering the particular disease.

The present invention also provides a method for diagnosing a disease, testing food, or assessing the environment by using the biomarker as described above. In the testing method with a biomarker, the biomarker is not required to be a peptide fragment determined by the above-mentioned screening method. As a biomarker, for example, a peptide fragment can be used that is obtained by subjecting a particular protein which has been purified to the fragmentation as described above. For diagnosis, a peptide fragment can be used that is prepared by protein engineering technique based on the gene sequence of a biomarker which has been obtained once. Also, a peptide fragment can be used that is synthesized on a peptide synthesizer or the like based on the amino acid sequence of a biomarker. The prepared biomarker can be used in diagnosis or testing, for example, as a competitive reagent for confirming that the binding of a biomarker in a sample with an affinity substance for detection is specific. Neither a method of screening for a biomarker nor a method for preparing a biomarker is limited to the above-mentioned methods. The present invention provides any biomarker-screening methods and any diagnosing methods using a biomarker which take advantage of the protein-fragmenting method according to the present invention. The biomarker-screening method and diagnosis method using a biomarker according to the present invention contribute to medical science, drug development, agriculture, and the like because, for example, they make it possible to use, as a biomarker, an insoluble protein or the like which was difficult to.

In a measurement method according to the present invention, a particular protein of interest is degraded into a particular peptide fragment not having an unstable conformation typical of protein, and the particular peptide fragment is measured mainly using immunoassay. Therefore it is possible that a protein of interest, regardless of whether it is soluble or insoluble, is measured with high stability and/or accuracy as compared with a conventional method, since uncertainty factors and/or disturbing factors, such as conformational structure or the like, are excluded from the measurement system. In addition, since a peptide fragment is a low molecular weight compound as compared with a protein and does not have an unstable conformation, it is possible to use a separation technique difficult to apply to high-molecular weight compound (for example, protein), such as liquid chromatography, in the measurement. Moreover, it is also possible that the measurement and storage are made under a wide range of conditions.

In a measurement method according to the present invention, it is not necessary to use an agent for maintaining solubility and therefore it is possible to even make such a measurement which is avoided to be made in the presence of the agent, such as UV absorption measurement, thereby broadening the choice of measurement system.

By a measurement method according to the present invention, even if a protein of interest is insoluble, it is possible to make a measurement in an aqueous environment (for example, an aqueous buffer solution system), especially in the same aqueous environment as one in which a soluble protein is measured. Therefore, it is possible to compare precisely the data of insoluble and soluble proteins under the same conditions. It is also possible to build a database including both insoluble and soluble protein data, which are preferably comparable with each other.

By a preparation method according to the present invention, it is possible to prepare a particular peptide fragment suitable for use in a measurement method according to the present invention, which takes advantage of competitive binding, from a particular protein (which is the final target for the measurement). It is also possible to use a particular peptide fragment prepared by this method, to produce and/or screen for an affinity substance specific for the original protein with high yield or efficiency, since the particular peptide fragment has little inhomogeneity factor due to, for example, the conformational structure of the original protein and/or the portions in the original protein other than the portion corresponding to the particular peptide fragment.

By a screening method according to the present invention, it is possible to use, as a biomarker, a portion (or fragment) of a protein which was conventionally difficult to as whole, such as insoluble protein.

As described above, by a method according to the present invention, it is possible to make a (precise) measurement of a protein which is difficult to by a conventional method. The present invention has significant advantages in the field of proteome analysis, medical science, drug development, agriculture, food, environment and the like.

### EXAMPLES

### Example 1

The measuring device according to the present invention in which a soluble peptide fragment to be detected can be measured with affinity electrophoresis was made, and mouse prion protein (as a particular protein) in the brain sample was measured using the device.

The mouse brain tissue containing mouse brain prion was homogenized with a beads-containing homogenization tube included in the Plateria BSE-kit (Bio-Rad Laboratories, USA) to prepare the 20% brain emulsion. The brain emulsion was diluted in water to prepare 20 μL of dilute emulsions containing each 600 μg, 200 μg, and 20 μg of brain tissue homogenate. To the dilute emulsions, 2 μL of 3 M sodium acetate and then 50 μL of 99.5% ethanol were added. After stirring, the dilute emulsions were allowed to stand at room temperature for five minutes. The dilute emulsions were centrifuged at 14,000 rpm for 10 minutes and the supernatants were discarded. The precipitates were suspended again in 70% ethanol containing 0.1 M sodium acetate, and then the suspensions were centrifuged again at 14,000 rpm for five minutes. The precipitates were dried with a centrifugal evaporator.

The residues were dissolved into 10 μL of 1 mM hydrochloric acid to prepare samples that had not been subjected to degradation into peptide fragments. Separately, to the residues, 20 μL of a solution of cyanogen bromide dissolved at 10 mg/mL in 70% formic acid was added at 50° C. for one hour, thereby performing degradation into peptide fragments. Cyanogen bromide and formic acid were evaporated with a centrifugal evaporator, and the residues were dissolved in 10 μL of 1 mM hydrochloric acid to prepare samples that had been subjected to degradation.

To 1 mL of both types of samples containing different concentrations of mouse prion, 9 μL of carrier ampholite for isoelectric focusing electrophoresis and 10 μL of 5×10⁻⁸ M anti-mouse prion single-chain antibody fragment that had been labeled with a fluorescent dye of tetramethylrhodamine were added to prepare a sample-loading solution used for affinity isoelectric focusing electrophoresis.

Next, the inner wall of a fused silica capillary (50 μm in inside diameter, 375 μm in outside diameter, and 18 cm in length) was coated with polydimethyl acrylamide to exclude the influence of the electroosmotic flow. Different capillaries were filled with each of the sample-loading solutions. Plastic vessels each including a platinum electrode were attached to the both ends of each capillary and used as an anolyte reservoir and a catholyte reservoir respectively. 20 mM phosphoric acid solution was used as anolyte and 20 mM sodium hydroxide solution as catholyte. A voltage was applied between the electrodes at electric field strength of 500 V/cm, and isoelectric focusing electrophoresis was performed out for 10 minutes. After completion of isoelectric focusing electrophoresis, the capillary was scanned for fluorescence by moving it to the detection point starting with the cathode (high pH) end. Fluorescence was excited with a green (534.5 nm) helium-neon laser (1 mW) and detected with a photoelectron multiplier through a band-pass filter having a center wavelength of 590 nm and a bandwidth of 40 nm.

In isoelectric focusing electrophoresis on the labeled antibody fragment alone, a single sharp peak of fluorescence was detected. The peak position is the isoelectric point position of the labeled antibody fragment.

The only peak was observed at the isoelectric point position of the labeled antibody fragment in affinity isoelectric focusing electrophoresis on all of the samples that had not been subjected to degradation. This indicates that prion protein was not able to be bound to the antibody in the measurement system used because prion protein is insoluble. In addition, there was a poor reproducibility of results. It is thought that this is the result of the presence of an insoluble protein(s) in the sample.

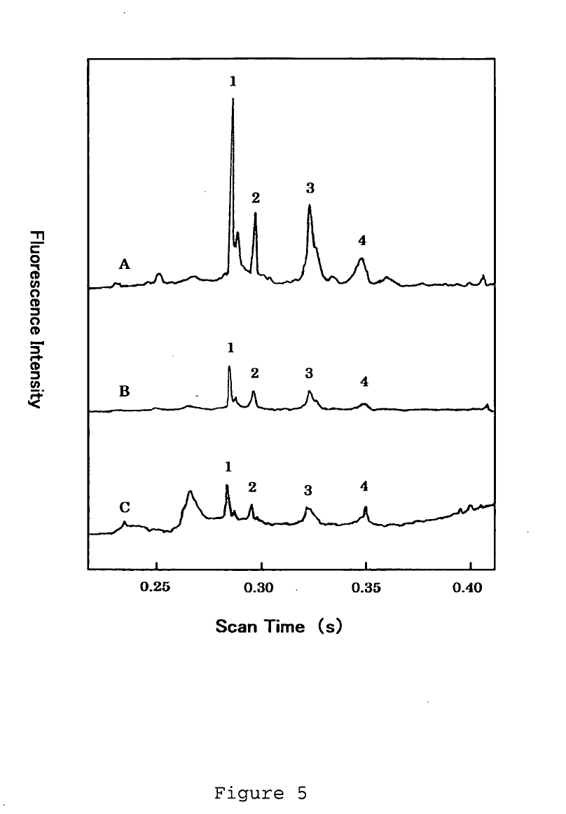

In contrast, as shown in FIG. 5, peaks were observed at positions different from the isoelectric point position of the labeled antibody fragment in affinity isoelectric focusing electrophoresis on all of the samples that had been subjected to degradation. It is thought that this is the result of particular soluble peptide fragments being generated by the degradation of prion. It is noted that the peak of unbound fluorescently labeled antibody is out of FIG. 5.

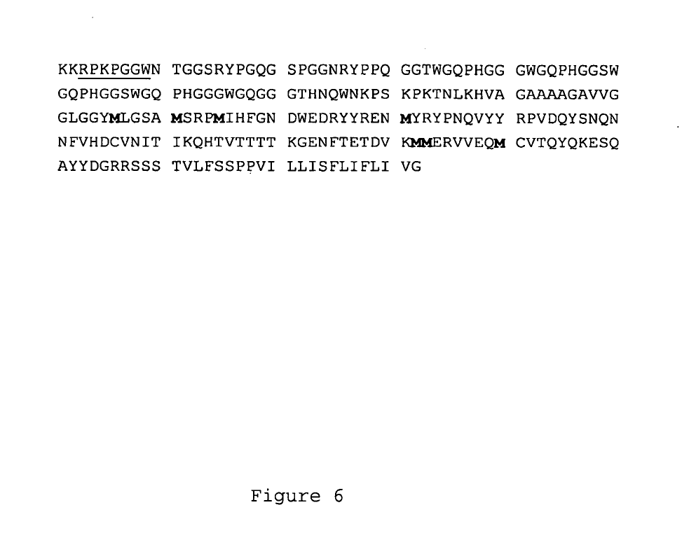

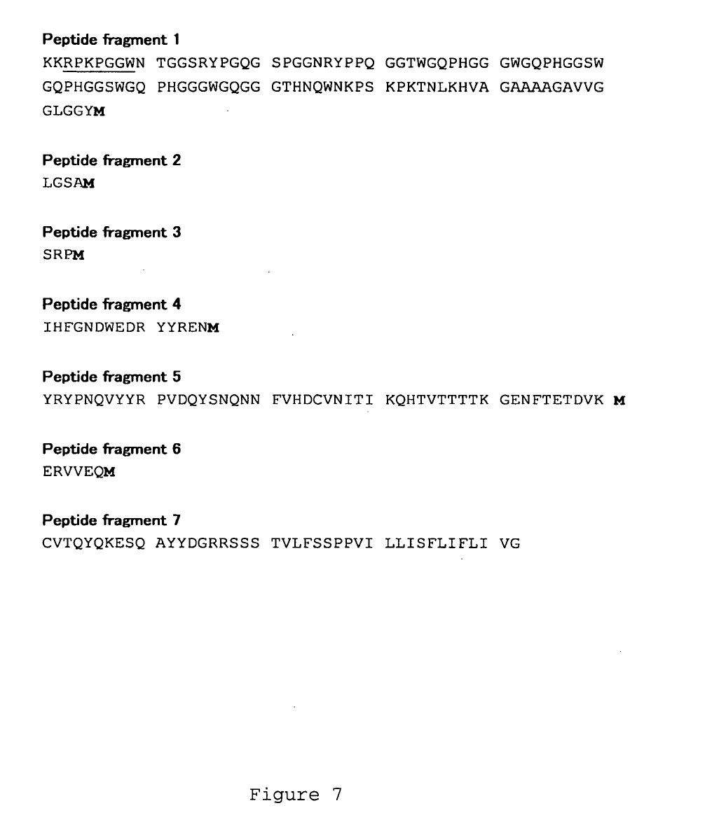

FIG. 6 shows the entire amino acid sequence of the prion protein from the mouse brain. Each of amino acids is represented by a single letter code. Because cyanogen bromide was used as a degrading reagent in this example, seven peptide fragments were generated from the prion protein from the mouse brain by cleaving the peptide bonds on C terminal side of methionine residues (“M” in FIG. 6). The amino acid sequences of the seven peptide fragments are shown in FIG. 7. It is known that the anti-mouse prion single-chain antibody fragment used recognizes the underlined amino acids as an affinity binding site (FIGS. 6 and 7). Therefore, it is thought that the anti-mouse prion single chain antibody fragment reacted to specifically bind and form a complex with peptide fragment 1 shown in FIG. 7. After the reaction, isoelectric focusing electrophoresis allowed the excess fluorescently labeled antibody to converge to the isoelectric point position of antibody alone, and the complex of peptide fragment 1 from mouse prion protein with the fluorescently labeled antibody to converge to the isoelectric point position of the complex. The converged positions were clearly different. Therefore, it is possible to measure the presence/absence of the antigen-antibody reaction, that is, the amount of peptide fragment 1. Because peptide fragment 1 is generated with cyanogens bromide from mouse prion protein in a proportional manner, there is a correlation between the amount of peptide fragment 1 and the amount of the original mouse prion protein. Therefore, the presence and the amount of mouse prion protein are indicated as the presence and the amount of peptide fragment 1.

As shown in FIG. 5, there are four peaks, indicating the presence of four complexes. Based on the amino acid sequence of peptide fragment 1, it is thought that this is because of the change in the isoelectric point of peptide fragment 1 by deamidation of asparagine residues (“N” in single letter code) and glutamine residues (“Q” in single letter code), or the presence/absence of a posttranslational modification of peptide fragment 1. If peptide fragment 1 is deamidated, it is possible to obtain a single peak due to a single complex by preserving the original brain sample that contains mouse prion protein under the optimal conditions to prevent the deamidation. Also, the concentration of mouse prion protein can be calculated, based on all of the peak information, i.e., from the total concentration of the four complexes. The presence/absence of a posttranslational modification can be determined by identification with MS.

When the above-mentioned experiment was repeated, there was a good reproducibility of results.

### Example 2

The measuring device according to the present invention in which a soluble peptide fragment to be detected can be measured by means of immunoassay coupled with fluid control was made, and mouse brain prion protein (as a particular protein) in the brain sample was measured using the device.

The mouse brain tissue containing mouse brain prion was homogenized with a beads-containing homogenization tube included in the Plateria BSE-kit (Bio-Rad Laboratories, USA) to prepare the 20% brain emulsion. This brain emulsion was diluted in water to prepare dilute emulsions containing brain tissue homogenates. To the dilute emulsions, 3 M sodium acetate and then 99.5% ethanol were added. After stirring, the dilute emulsions were allowed to stand at room temperature for five minutes. The dilute emulsions were centrifuged at 14,000 rpm for 10 minutes and the supernatants were discarded. The precipitates were suspended again in 70% ethanol containing 0.1 M sodium acetate, and then the suspensions were centrifuged again at 14,000 rpm for five minutes. The precipitates were dried with a centrifugal evaporator.

The residues were dissolved into 1 mM hydrochloric acid to prepare samples that had not been subjected to degradation. Separately, to the residues, a solution of cyanogen bromide dissolved at 10 mg/mL in 70% formic acid was added at 50° C. for one hour, thereby performing degradation into peptide fragments. Cyanogen bromide and formic acid were evaporated with a centrifugal evaporator, and the residues were dissolved in 1 mM hydrochloric acid to prepare samples that had been subjected to degradation.

Then, the measuring device, which consists only of measurement member, was formed on a plastic substrate by milling. The measurement member is in a size of 3 mm×3 mm×1 mm. Three measuring devices were made in total.

Next, besides the above-mentioned samples, a known concentration of mouse prion protein was degraded with cyanogens bromide to generate peptide fragments. The peptide fragments were introduced into the measurement member of each measuring device and immobilized by adsorbing. A pipetter was used for the introduction. The measurement members were blocked by using BSA as a blocking agent, and then washed. Thus, three measuring devices, on the measurement members of which the known concentration of the peptide fragments of mouse prion protein were immobilized, were made.

A known concentration of anti-mouse prion single-chain antibody fragment that had been fluorescently labeled with tetramethylrhodamine was introduced into the measurement member of one of the measuring devices to allow for binding reaction. After washing, fluorescence was excited with a green (534.5 nm) helium-neon laser (1 mW) and measured with a photoelectron multiplier through a band-pass filter having a center wavelength of 590 nm and a bandwidth of 40 nm. Thus, information on fluorescence intensity was obtained in the absence of the peptide fragments from mouse prion protein.

Next, a known concentration of the single-chain antibody fragment was added and mixed to the sample that had not been subjected to degradation. The mixture was introduced into the measurement member in another measuring device. After washing, the fluorescence was measured. This fluorescence intensity was essentially the same as that in the absence of the peptide fragments from mouse prion protein. It is thought that this is because the sample had not been subject to degradation and thus prion remained insoluble, as a result the added antibody fragment was not bound before the introduction into the measurement member but after the introduction, the added antibody fragment was all bound to the peptide fragments immobilized on the measurement member.

Next, a known concentration of the single-chain antibody fragment was added and mixed to the sample that had been subjected to degradation. The mixture was introduced into the measurement member in the third one of the measuring devices. After washing, the fluorescence was measured. This fluorescence intensity was decreased as compared with that in the absence of the peptide fragments from mouse prion protein. It is thought that this is because the sample had been subject to degradation and thus prion had been degraded into peptide fragments to generate a soluble peptide fragment having a binding site for the labeled single-chain antibody fragment, as a result the added antibody fragment was bound to the soluble peptide fragment before the introduction into the measurement member and accordingly the amount of the unbound antibody fragment which have been, in turn, introduced into the measurement member was decreased.

In conclusion, it is indicated that the method according to the present invention makes it possible to measure of mouse prion protein, which was impossible to measure by the conventional method because mouse prion protein is insoluble in itself. Because the amount of the peptide fragment is proportional to that of the original protein, the latter can be determined by measuring the former.

When the present experiment was repeated, there was a good reproducibility of results.

The detailed description provided above, however, merely illustrates the principles of the invention. It will thus be appreciated that those skilled in the art will be able to devise various arrangements which, although not explicitly described or shown herein, embody the principles of the invention and are thus within its spirit and scope.

1. A method for measuring a particular protein in a sample containing at least one protein, wherein the sample is reacted with a reagent cleaving a peptide bond of the particular protein to generate a soluble peptide fragment which is determined by a certain primary structure; and contacted with a reagent reacting specifically with the particular soluble peptide fragment, thereby detecting the presence of the particular soluble peptide fragment.

2. The method according to claim 1, wherein the measurement of the presence of the soluble peptide fragment to be detected is conducted by separating a complex which is formed by contacting between the soluble peptide fragment to be detected and the reagent reacting specifically with the particular soluble fragment, from the peptide fragments and the reagent which are not formed the complex.

3. The method according to claim 1, wherein the reagent cleaving a peptide bond of the particular protein is a regent cleaving a protein at a site of a certain amino acid or amino acid sequence.

4. The method according to claim 3, wherein the regent cleaving a protein at a site of a certain amino acid or amino acid sequence is a reagent using an enzymatic reaction.

5. The method according to claim 3, wherein the regent cleaving a protein at a site of a certain amino acid or amino acid sequence is a reagent using a chemical reaction.

6. The method according to claim 4, wherein the reagent using an enzymatic reaction is a protease.

7. The method according to claim 6, wherein the protease is selected from the group consisting of trypsin, chymotrypsin, pepsin, brornelain, elastase, clostripain, V8-protease, thermolysin, lysyl endopeptidase, arginine endopeptidase, prolyl endopeptidase, and aspartic acid-N protease.

8. The method according to claim 5, wherein the reagent using a chemical reaction is selected from the group consisting of cyanogen bromide, Ntromosuccinimide, BNPS-skatole, dimethyl sulfoxide-HG1-HBr, iodosylbenzoicacid, N-chlorosuccinimide, hydroxylamine and guanidine hydrochloride.

9. The method according to claim 1, wherein the reagent reacting specifically with the particular soluble peptide fragment to be detected is an affinity substance having a binding affinity for the particular protein.

10. The method according to claim 1, wherein the reagent reacting specifically with the particular soluble peptide fragment to be detected is an affinity substance having a binding affinity for the particular soluble peptide fragment to be detected.

11. The method according to claim 9, wherein the affinity substance is selected from the group consisting of proteins, peptides, nucleic acids and synthetic chemicals.

12. The method according to claim 1, wherein the reagent reacting specifically with the particular soluble peptide fragment to be detected has a label for the measurement.

13. The method according to claim 12, wherein the label for the measurement is selected from the group consisting of fluorescent dyes, enzymes, absorbing pigments, chemiluminophores, radioisotopes, spin labels and electrochemical labels.

14. The method according to claim 12, wherein the measurement of the label is by photodetection.

15. The method according to claim 12, wherein the measurement of the label is by electrical measurement.

16. The method according to claim 1, wherein the measurement of the presence of the soluble peptide fragment to be detected is by immunoassay.

17. The method according to claim 16, wherein the immunoassay is affinity electrophoresis.

18. The method according to claim 17, wherein the affinity electrophoresis is affinity isoelectric focusing electrophoresis.

19. The method according to claim 16, wherein the immunoassay is immunoassay coupled with fluid control.

20. The method according to claim 1, wherein the particular protein is a membrane protein

21. The method according to claim 1, wherein the particular protein is prion protein.

22. The method according to claim 1, wherein the presence of the soluble peptide fragment to be detected is measured quantitatively.

23. A device for use in the method according to claim 18, comprising a flow channel in which the affinity isoelectric focusing electrophoresis is performed, an anolyte reservoir which is filled with anolyte, and a catholyte reservoir which is filled with eatholyte.

24. The device according to claim. 23, wherein the anolyte reservoir and the catholyte reservoir each include an electrode or has a mechanism for holding an electrode inserted outside.

25. The device according to claim 24, further comprising a mechanism for applying a voltage between the electrodes to perform the electrophoresis.

26. The device according to claim 23, wherein the width and depth of the flow channel are in the range of 1 μm to 5000 μm, respectively.

27. A device for use in the method according to claim 19, wherein comprising a measurement member for measuring the presence/absence and the concentration of the soluble peptide fragment to be detected with immunoassay coupled with fluid control.

28. The device according to claim 27, further comprising an introduction member, a drainage member, a flow channel connecting between the measurement member and the introduction member, and another flow channel connecting between the measurement member and the drainage member.

29. The device according to claim 27, further comprising a solution feeding system for introducing and draining a solution.

30. The device according to claim 23, further comprising a detection device for detecting the reagent which is bound to the soluble peptide fragment to be detected.

31. The device according to claim 30, wherein the detection device is a photodetecion device, which comprises a light source selected from the group consisting of lasers or LEDs, or lamps and a detector selected from the group consisting of photoelectron multipliers and multipixel photodetectors.

32. The device according to claim 31, wherein the light from the light source is introduced from one of the ends of the flow channel.

33. A method for preparing a peptide fragment which is capable of being bound by a substance having an affinity for a particular protein, wherein a protein preparation containing the particular protein is reacted with a reagent cleaving a protein at a site of a certain amino acid or amino acid sequence to generate a soluble peptide fragment which is determined by a certain primary structure; and contacted with a reagent reacting specifically with the particular soluble peptide fragment, thereby collecting the particular soluble peptide fragment.

34. The method according to claim 33, wherein the reagent cleaving a protein at a site of a certain amino acid or amino acid sequence is a reagent using an enzymatic reaction.

35. The method according to claim 33, wherein the regent cleaving a protein at a site of a certain amino acid or amino acid sequence is a reagent using a chemical reaction.

36. The method according to claim 34, wherein the reagent using an enzymatic reaction is a protease.

37. The method according to claim 36, wherein the protease is selected from the group consisting of trypsin, chymotrypsin, pepsin, bromelain, elastase, clostripain, V8-protease, thermolysin, lysyl endopeptidase, arginine endopeptidase, prolyl endopeptidase and aspartic acid-N protease.

38. The method according to claim 35, wherein the reagent using a chemical reaction is selected from the group consisting of cyanogens bromide, N-bromosuccinimide, BNPS-skatole, dimethyl sulfoxide-HCl-HBr, iodosylbenzoic acid, N-chlorosuccinimide, hydroxylamine and guanidine hydrochloride.

39. A method for screening for a biomarker, wherein a sample containing at least one protein is reacted with a reagent cleaving a protein at a site of a certain amino acid or amino acid sequence to generate soluble peptide fragments which is determined by a certain primary structure; and the soluble peptide fragments are screened for the biomarker.

40. The method according to claim 39, wherein the reagent cleaving a protein at a site of a certain amino acid or amino acid sequence is a reagent using an enzymatic reaction.

41. The method according to claim 39, wherein the regent cleaving a protein at a site of a certain amino acid or amino acid sequence is a reagent using a chemical reaction.

42. The method according to claim 40, wherein the reagent using an enzymatic reaction is a protease.

43. The method according to claim 42, wherein the protease is selected from the group consisting of trypsin, chymotrypsin, pepsin, bromelain, elastase, clostripain, V8-protease, thermolysin, lysyl endopeptidase, arginine endopeptidase, prolyl endopeptidase and aspartic acid-N protease.

44. The method according to claim 41, wherein the reagent using a chemical reaction is selected from the group consisting of cyanogens bromide, N-bromosuccinimide, BPNS-skatole, dimethyl sulfoxide-HCl-HBr, iodosylbenzoic acid, N-chlorosuccinimide, hydroxylamine and guanidine hydrochloride.

45. The method according to claim 39, wherein the screening for the biomarker is by immunoassay.

46. The method according to claim 45, wherein the immunoassay is affinity electrophoresis.

47. The method according to claim 46, wherein the electrophoresis is affinity isoelectric focusing electrophoresis.

48. The method according to claim 45, wherein the immunoassay is immunoassay coupled with fluid control.

49. A testing method for measuring in a sample the presence of the biomarker determined by the method according to claim 39.


SEQUENCE LISTING (8 sequences)

SEQ ID NO: 1 - protein, 232 aa, mouse
KKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 2 - protein, 106 aa, mouse
KKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYM

SEQ ID NO: 3 - protein, 5 aa, mouse
LGSAM

SEQ ID NO: 4 - protein, 4 aa, mouse
SRPM

SEQ ID NO: 5 - protein, 16 aa, mouse
IHFGNDWEDRYYRENM

SEQ ID NO: 6 - protein, 51 aa, mouse
YRYPNQVYYRPVDQYSNQNNFVHDCVNITIKQHTVTTTTKGENFTETDVKM

SEQ ID NO: 7 - protein, 7 aa, Mougeotia scalaris
ERVVEQM

SEQ ID NO: 8 - protein, 42 aa, mouse
CVTQYQKESQAYYDGRRSSSTVLFSSPPVILLISFLIFLIVG